(19) United States
(12) Patent Application Publication
(10) Pub. No.: US 2021/0254174 A1
(43) Pub. Date: Aug. 19, 2021

(54) LIVER CANCER-SPECIFIC BIOMARKER

(71) Applicant: GECKO BIOLAB, Seoul (KR)

(72) Inventors: Suk Woo NAM, Seoul (KR); Jung Woo EUN, Suwon-si (KR); Jeong Won JANG, Seoul (KR)

(73) Assignee: GECKO BIOLAB, Seoul (KR)

(21) Appl. No.: 17/252,066

(22) PCT Filed: Jun. 13, 2019
(86) PCT No.: PCT/KR2019/007131

§ 371 Date: Dec. 14, 2020

(30) Foreign Application Priority Data
Jun. 14, 2018 (KR) 10-2018-0067968

(51) Int. Cl. C12Q 1/6886; G01N 33/574

(52) U.S. Cl. CPC C12Q 1/6886; C12Q 2600/158; G01N 33/57438

(57) ABSTRACT

The present disclosure relates to the use of genes whose expression or protein changes specifically to hepatocellular carcinoma as biomarkers for the detection and diagnosis of hepatocellular carcinoma, in which the biomarkers of the present disclosure, HMMR, NXPH4, PITX1, THBS4, and UBE2T, since they change their expression specifically to hepatocellular carcinoma, may be used as hepatocellular carcinoma-specific markers, and furthermore, these biomarkers may be used independently or in combination with AFP, or may be independently combined to make a more specific and accurate diagnosis of hepatocellular carcinoma.

### CROSS REFERENCE TO RELATED APPLICATION

This application is a National Stage of International Application No. PCT/KR2019/007131 filed Jun. 13, 2019, claiming priority based on Korean Patent Application No. 10-2018-0067968 filed Jun. 14, 2018, the entire disclosures of which are incorporated herein by reference.

### TECHNICAL FIELD

The present disclosure relates to a liver cancer-specific biomarker, and more particularly, to uses of genes and proteins whose expression changes specifically to hepatocellular carcinoma as biomarkers for detection and diagnosis of hepatocellular carcinoma.

### BACKGROUND ART

Among cancers, liver cancer is known as one of the deadliest cancers in the world. In particular, it is reported that more than 500,000 people die of liver cancer each year in Asia and sub-Saharan Africa. Liver cancer may be divided into primary liver cancer (hepatocellular carcinoma) arising from liver cells itself and metastatic liver cancer in which cancers of other tissues have spread to the liver. More than 90% of liver cancers are primary liver cancer.

The hepatocellular carcinoma (HCC) is the 5th most common tumor in the world which kills 500,000 people annually (Okuda 2000). The survival rate of HCC patients has not improved over the past 20 years and has an incidence rate that is almost identical to the mortality rate (Marrero, Fontana et al 2005). Chronic hepatitis caused by infection with hepatitis B virus or hepatitis C virus and exposure to carcinogens such as aflatoxin B1 are known as major risk factors for HCC (Thorgeirsson and Grisham 2002). It is reported that changes in cell cycle regulators that proceed to the G1 stage in the cell cycle mechanism are related to the formation of liver cancer (Hui et al, Hepatogasteroenterology 45:1635-1642, 1998). However, the intracellular molecular mechanisms involved in the onset and progression of liver cancer have not yet been clearly identified. According to a conventional study, it has been reported that when a protooncogene such as various growth factor genes is mutated into oncogene and thus is overexpressed or has overactivity due to various causes, or when a tumor suppressor gene such as Rb protein or p53 protein is mutated and thus has underexpression or loss of function due to various causes, the onset and progression of various cancers including liver cancer are caused. In addition, it is reported that DNA mutation and genetic alteration of gene expression are identified in liver cancer patient tissues (Park et al, Cancer Res 59:307-310, 1999; Bjersing et al, J Intern Med 234:339-340, 1993; Tsopanomichalou et al, Liver 19:305-311, 1999; Kusano et al, Hepatology 29: 1858-1862, 1999; Keck et al, Cancer Genet Cytogenet 111:37-44, 1999). Recently, it is recognized that the onset and progression of most cancers, including liver cancer, is not caused by a few specific genes, but is caused by complex interactions of various genes related to cell cycle and signaling. Therefore, the need for comprehensive research on various genes or proteins is emerging, rather than focusing only on the expression or function of individual genes or proteins.

Further, a biomarker test that may detect liver cancer early and accurately in normal people has not yet been developed. The serum alpha-fetoprotein (AFP) test is used to diagnose non-invasive early liver cancer in high-risk groups. At the time of development of AFP, a reference value thereof that may achieve both sensitivity and specificity at a good level was suggested as 20 ng/mL. In this case, the sensitivity is only 60%. When the liver cancer is diagnosed based on 200 ng/mL thereof according to the current international society's liver cancer diagnosis guidelines, the specificity increases, but the sensitivity is only 22%. As a result of previous studies, AFP is known to have a sensitivity of about 66% and a specificity of 82%, and has limitation in diagnosing all liver cancer patients. Serum markers that are not established as diagnostic standards but helpful in diagnosing liver cancer include Descarboxyprothrombin (DCP), Prothrombin Induced by Vitamin K Absence II (PIVKA-II), glycosylated AFP versus total AFP (L3 fraction) distribution, alpha fucosidase, glypican 3, HSP-70, and the like. However, they have meaning only as a prognostic factor. When used alone, the accuracy thereof is low and each thereof has not yet been used for a screening test. Early diagnosis of liver cancer may be difficult. In addition, patients diagnosed to have the liver cancer at the stage where radical treatments such as actual surgery or high-frequency heat therapy are possible are limited to around 30% of all liver cancer patients.

### DISCLOSURE

### Technical Purpose

The present disclosure aims to develop new diagnostic markers with improved specificity and sensitivity by which the liver cancer may be diagnosed as early as possible at the stage where fundamental treatment of liver cancer is possible.

### Technical Solution

To achieve the above purpose, the present disclosure provides a biomarker for diagnosis of liver cancer.

Further, the present disclosure provides a composition for diagnosis of liver cancer.

Further, the present disclosure provides a liver cancer diagnostic kit.

In addition, the present disclosure provides a method of providing information necessary for liver cancer diagnosis.

### Advantageous Effects

According to the present disclosure, the expression of HMMR (hyaluronan-mediated motility receptor), NXPH4 (neurexophilin 4), PITX1 (paired-like homeodomain 1), THBS4 (thrombospondin 4), or UBE2T (ubiquitin-conjugating enzyme E2T) as the biomarker according to the present disclosure changes specifically to hepatocellular carcinoma, and thus may be used as hepatocellular carcinoma-specific markers. These may be used alone or in combination with each other or in combination with AFP (a-fetoprotein), to achieve the effect of more specific and accurate diagnosis of hepatocellular carcinoma.

### DESCRIPTION OF DRAWINGS

FIG. 1 is a diagram showing the designed cohort studies to identify hepatocellular carcinoma-specific markers, and clinical validate with liver cancer patients according to the present disclosure.

FIG. 2 is a diagram showing a process of deriving 2,502 gene-elements having transmembrane domain that are overexpressed, and that are secretory peptide or protein in hepatocellular carcinoma cells. FIG. 3 is a diagram showing the results of analyzing Cancer Genome Atlas hepatocellular carcinoma (TCGA_LIHC) data and Gene Expression Omnibus (GEO) database and thus hierarchical clustering analysis of 737 genes overexpressed in both databases.

FIG. 4A to FIG. 4D are diagrams identifying 10 candidate marker genes that appear to be gradually overexpressed in the development of multistage liver cancer in both the GSE114564 and GSE6764 data cohort.

FIG. 5A to FIG. 5D show the results of differential gene expressions of candidates 10 marker genes in HCC patients with corresponding non-cancer derived from the TCGA_LIHC and ICGC_LIRI data sets, respectively.

FIG. 6A to FIG. 6B show the results of differential gene expressions of candidates 10 marker genes in matched-pairs of HCC patients in the GSE77314 data set.

FIG. 7 shows blood serum levels of AFP in the test cohort including total 100 of chronic liver disease, cancer patients, and healthy normal by ELISA.

FIG. 8A to FIG. 8B show the result of blood serum levels of each maker of 10 tested markers in the chronic liver disease, cancer patients, and healthy normal by ELISA.

FIG. 9A to FIG. 9B shows the results of ROC curve analysis with ELISA values for 10 marker genes.

FIG. 10 blood serum levels of AFP in the validation (independent liver disease patients) cohort (1,148 samples from 279 patients) including chronic liver disease, cancer patients, and healthy normal, and also shows ROC curve analysis with ELISA.

FIG. 11A to FIG. 11B show the results of blood serum levels of each maker of 5 selected markers in the chronic liver disease, cancer patients, and healthy normal by ELISA.

FIG. 12A to FIG. 12B show the results of ROC curve analysis with ELISA values for 5selected marker proteins HMMR, NXPH4, PITX1, THBS4 and UBE2T.

FIG. 13A to FIG. 13B shows positive and negative rates of indicated markers including AFP, HMMR, NXPH4, PITX1, THBS4 and UBE2T in NL (healthy normal liver), CH (chronic hepatitis), LC (liver cirrhosis), eHCC (early hepatocellular carcinoma), avHCC (advanced hepatocellular carcinoma) patients groups, respectively.

FIG. 13C shows comparative analysis of ROC curves of 5 markers, HMMR, NXPH4, PITX1, THBS4, UBE2T, and AFP, as standard HCC marker, in non-tumor vs HCC or chronic liver disease (CH, LC) vs HCC, respectively.

FIG. 13D shows positive and negative rates of indicated markers including AFP, HMMR, NXPH4, PITX1, THBS4 and UBE2T in patients with or without AFP positive in HCC patients.

FIG. 13E shows comparative analysis of ROC curves of 5 markers, HMMR, NXPH4, PITX1, THBS4, UBE2T, and AFP, as standard HCC marker, in non-tumor vs early HCC (eHCC) or chronic liver disease (CH, LC) vs eHCC, respectively.

FIG. 13F shows positive and negative rates of indicated markers including AFP, HMMR, NXPH4, PITX1, THBS4 and UBE2T in patients with or without AFP positive in early HCC (eHCC) patients.

FIG. 13G shows positive rates of indicated marker including 5 markers, HMMR, NXPH4, PITX1, THBS4, UBE2T, and AFP, as standard HCC marker in total 132 HCC patients and 69 early HCC (eHCC) patients, respectively.

FIGS. 14A and 14B show diagnostic accuracies of the makers indicated combinations with or without AFP in HCC (FIG. 14A) and early HCC (FIG. 14B), respectively.

FIG. 14C shows ROC curve of AFP, or combination of two markers for patients with all HCC (n=132) versus all controls (n=86). ROC curve of AFP, or combination of three markers for patients with all HCC (n=132) versus all controls (n=86).

FIG. 14D shows ROC curve of AFP, or combination of two markers for patients with early-stage HCC (n=69) versus all controls (n=86). ROC curve of AFP, or combination of three markers for patients with early-stage HCC (n=69) versus all controls (n=86).

### MODES OF THE INVENTION

Hereinafter, the present disclosure will be described in detail based on an embodiment of the present disclosure with reference to the accompanying drawings. However, the following embodiment is presented as an example of the present disclosure. When it is determined that a detailed description of a well-known technology or configuration known to those skilled in the art may unnecessarily obscure the subject matter of the present disclosure, the detailed description may be omitted. This omission does not limit a scope of the present disclosure. The present disclosure may be variously modified and applied within an equivalent scope interpreted from the description of the claims to be described later.

Further, terms used in this specification are terms used to properly describe a preferred example of the present disclosure, and may vary depending on the intention of users or operators, or customs in the field to which the present disclosure belongs. Accordingly, definitions of these terms should be made based on the contents in the present specification. Throughout the specification, when a portion “includes” a certain component, it means that other components may be further included therein rather than excluding other components unless specifically otherwise stated.

Unless otherwise indicated, nucleic acids are written in a 5′→3′ orientation from left to right. Numerical ranges listed within the specification include numbers defining the ranges. Each integer or any non-integer fraction within the defined range is included therein.

Unless otherwise defined, all technical and scientific terms used herein have the same meaning as commonly understood by one of ordinary skill in the art to which the present disclosure belongs. While any methods and materials similar or equivalent to those described herein may be used in practice to test the present disclosure, preferred materials and methods are described herein.

In the present disclosure, the term “subject” or “patient” refers to any single individual requiring treatment, including humans, apes, monkeys, cows, dogs, guinea pigs, rabbits, chickens, insects, and the like. Further, the subject includes any subject who participates in a clinical research trial and does not show any disease clinical findings, or who participates in an epidemiological study or a subject used as a control.

In the present disclosure, the term “sample” refers to a biological sample obtained from a subject or patient. The source of the biological sample may be a fresh, frozen and/or preserved organ or tissue sample or solid tissue from a biopsy or aspirate; blood or any blood component; a cell at any point in the subject's pregnancy or development. In one example according to the present disclosure, blood or any blood component was taken as a sample.

All technical terms used in the present disclosure, unless otherwise defined, are used in the same meaning as those of ordinary skill in the art generally understand in the related field of the present disclosure. In addition, although preferred methods or samples are described in the present specification, similar or equivalent ones are included in the scope of the present disclosure. The contents of all publications referred to herein by reference are incorporated into the present disclosure.

In one aspect, the present disclosure relates to a biomarker for diagnosis of liver cancer including at least one gene selected from a group consisting of AFP (α-fetoprotein), HMMR (hyaluronan-mediated motility receptor), NXPH4 (neurexophilin 4), PITX1 (paired-like homeodomain 1), THBS4 (thrombospondin 4) and UBE2T (ubiquitin-conjugating enzyme E2T) or a protein expressed from the at least one gene. In one example of the present disclosure, a schematic diagram of the sequence of identification of the liver cancer-specific biomarkers is shown in FIG. 1.

In one embodiment, the liver cancer may be hepatocellular carcinoma (HCC), and may be early hepatocellular carcinoma or advanced hepatocellular carcinoma.

In one embodiment, the expression of the biomarker genes in accordance with the present disclosure may be increased specifically to liver cancer.

In one aspect, the present disclosure relates to a composition for diagnosis of liver cancer, the composition including an agent for measuring an expression level of one or more biomarker genes selected from a group consisting of AFP, HMMR, NXPH4, PITX1, THBS4 and UBE2T at an mRNA or protein level.

In one embodiment, the composition may include an agent for measuring an expression level of one or more biomarker gene sets selected from a group consisting of AFP and HMMR, AFP and NXPH4, AFP and PITX1, AFP and THBS4, AFP and UBE2T, HMMR and NXPH4, HMMR and PITX1, HMMR and THBS4, HMMR and UBE2T, NXPH4 and PITX1, NXPH4 and THBS4, NXPH4 and UBE2T, PITX1 and THBS4, PITX1 and UBE2T, and THBS4 and UBE2T at the mRNA or protein level.

In one embodiment, the composition may include an agent for measuring an expression level of one or more biomarker gene sets selected from a group consisting of AFP, HMMR and NXPH4; AFP, HMMR and PITX1; AFP, HMMR and THBS4; AFP, HMMR and UBE2T; AFP, NXPH4 and PITX1; AFP, NXPH4 and THBS4; AFP, NXPH4 and UBE2T; AFP, PITX1 and THBS4; AFP, PITX1 and UBE2T; AFP, THBS4 and UBE2T; HMMR, NXPH4 and PITX1; HMMR, NXPH4 and; HMMR, NXPH4 and THBS4; HMMR, NXPH4 and UBE2T; HMMR, PITX1 and THBS4; HMMR, PITX1 and UBE2T; HMMR, THBS4 and UBE2T; NXPH4, PITX1 and THBS4; NXPH4, PITX1 and UBE2T; and NXPH4, THBS4 and UBE2T at the mRNA or protein level.

In one embodiment, the agent for measuring the expression level of the biomarker gene at the mRNA level may include a nucleic acid sequence of the marker, a nucleic acid sequence complementary to the nucleic acid sequence, and a primer pair and/or a probe that specifically recognizes a fragment of the nucleic acid sequence and the complementary nucleic acid sequence. Measurements thereof may be performed using a scheme selected from a group consisting of polymerase chain reaction, real-time RT-PCR, reverse transcription polymerase chain reaction, competitive polymerase chain reaction (competitive RT-PCR), nuclease protection assay (RNase, S1 nuclease assay), in situ hybridization, nucleic acid microarray, Northern blots, and DNA chip.

In one embodiment, the agent for measuring the expression level of the biomarker gene at the protein level may include an antibody, an antibody fragment, an aptamer, an avidity multimer or peptidomimetics that specifically recognizes a full length of a protein of the maker or a fragment thereof. Measurements thereof may be performed using a scheme selected from a group consisting of Western blot, ELISA (enzyme linked immunosorbent assay), radioimmunoassay (RIA), radioimmunodiffusion, immunoelectrophoresis, tissue immunostaining, immunoprecipitation assay, complement fixation assay, FACS, mass spectrometry, and protein microarray.

The term “detection” or “measurement” as used in the present disclosure means quantifying the concentration of a detection or measurement target.

In the present disclosure, the term “primer” refers to a nucleic acid sequence with a short free 3 hydroxyl group and means a short nucleic acid sequence which may form a base pair together with a complementary template thereto and may serve as a starting point for template strand copying. The primer may initiate DNA synthesis in the presence of a reagent for polymerization (that is, DNA polymerate or reverse transcriptase) and four different nucleoside triphosphates in an appropriate buffer solution and temperature.

In the present disclosure, the term “probe” refers to a nucleic acid fragment of RNA or DNA corresponding to several bases to hundreds of bases capable of achieving specific binding to mRNA. The probe may be labeled to identify the presence or absence of a specific mRNA. The probe may be manufactured in the form of an oligonucleotide probe, a single stranded DNA probe, a double stranded DNA probe, or an RNA probe. In the present disclosure, hybridization between AFP, HMMR, NXPH4, PITX1, THBS4 and/or UBE2T gene and a probe complementary thereto may be performed such that the gene expression level may be diagnosed based on absence or presence of hybridization. Since the selection of the appropriate probe and the hybridization condition may be modified based on those known in the art, the present disclosure is not particularly limited thereto.

The primer or the probe according to the present disclosure may be chemically synthesized using the phosphoramidite solid support method, or other well known methods. Such nucleic acid sequences may also be modified using a number of means known in the art. Non-limiting examples of such modifications include methylation, encapsulation, substitution with one or more homologs of natural nucleotides, and modifications between nucleotides, for example, modifications to uncharged linkers (e.g., methyl phosphonate, phosphotriester, phosphoroamidate, carbamate, etc.) or charged linkers (e.g. phosphorothioate, phosphorodithioate, etc.).

In the present disclosure, suitable conditions for hybridizing a probe with a cDNA molecule may be determined in a series of steps via an optimization procedure. This procedure is performed in a series of procedures by a person skilled in the art to establish a protocol for use in a laboratory. For example, conditions such as temperature, concentration of components, hybridization and washing time, buffer components and pH and ionic strength thereof may depend on various factors such as the probe length and the GC amount and target nucleotide sequence. Detailed conditions for hybridization may be disclosed in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999). For example, among the stringent conditions, high stringency conditions are as follows: hybridization in 0.5 M NaHPO₄, 7% sodium dodecyl sulfate (SDS), 1 mM EDTA and at 65° C., and washing at 68° C. in 0.1×SSC (standard saline citrate)/0.1% SDS. Alternatively, high stringency conditions are as follows: washing at 48° C. in 6×SSC/0.05% sodium pyrophosphate. Low stringency conditions are as follows: washing at 42° C. in 0.2×SSC/0.1% SDS, for example.

In the present disclosure, the term “antibody” is a term known in the art and refers to a specific protein molecule directed against an antigenic site. For the purpose of the present disclosure, an antibody refers to an antibody that specifically binds to a protein expressed in the AFP, HMMR, NXPH4, PITX1, THBS4 and/or UBE2T gene, which are markers according to the present disclosure. The antibody preparation method may be a well-known method. The antibody includes partial peptides that may be made from the protein. The form of the antibody according to the present disclosure is not particularly limited. A polyclonal antibody, a monoclonal antibody, or one having antigen binding or a portion thereof is included in the antibody according to the present disclosure. All immunoglobulin antibodies are included therein. Furthermore, the antibodies according to the present disclosure include special antibodies such as humanized antibodies.

In one aspect, the present disclosure relates to a liver cancer diagnostic kit including the composition for diagnosing liver cancer.

In one embodiment, the kit may further include tools and/or reagents for collecting biological samples from a subject or patient, as well as tools and/or reagents for preparing genomic DNA, cDNA, RNA, or proteins from the samples. For example, the kit may contain PCR primers for amplifying the relevant region of genomic DNA. The kit may contain probes of genetic factors useful for pharmacogenomic profiling. Further, in the use of such a kit, easy identification may be carried out during analysis using a labeled oligonucleotide.

In one embodiment, the kit may further contain a labeling material such as a fluorescent material, DNA polymerase and dNTP (dGTP, dCTP, dATP and dTTP), etc.

In the present disclosure, the term “liver cancer diagnostic kit” refers to a kit including a composition for diagnosis of liver cancer in accordance with the present disclosure. Accordingly, the expression “liver cancer diagnostic kit” may be used interchangeably with “liver cancer diagnostic composition”. In this specification, the term “diagnosis” refers to determining the susceptibility of a subject to a specific disease or condition, determining whether a subject currently has a specific disease or condition, determining the prognosis (e.g., identification of a pre-metastatic or metastatic cancer state, determining the stage of the cancer, or determining the cancer's responsiveness to treatment) of a subject with a particular disease or condition, or therametrics (e.g., monitoring the condition of a subject to provide information about treatment efficacy).

In the present disclosure, the term “diagnosis biomarker, diagnostic biomarker, or diagnostic marker” refers to a substance which may distinguish the presence of liver cancer cells or tissues from normal cells or tissues to diagnose the liver cancer. The substance may include organic biomolecules, etc. such as polypeptides or nucleic acids (e.g. mRNA, etc.), lipids, glycolipids, glycoproteins, sugars (monosaccharides, disaccharides, oligosaccharides, etc.) that exhibit an increase or decrease in expression thereof in cells or tissues with liver cancer cells compared to normal cells. For the purpose of the present disclosure, the liver cancer detection or diagnosis biomarker includes at least one selected from a group consisting of genes AFP, HMMR, NXPH4, PITX1, THBS4 and UBE2T, as a gene whose mRNA expression or protein expression level increases specifically to liver cancer. These markers include not only the genes, but also DNA or mRNA that is complementary to any one marker. It is preferable that the marker is a complex marker containing two or more of the above markers. More preferably, the marker may include at least one selected from a group consisting of AFP and HMMR; AFP and NXPH4; AFP and PITX1; AFP and THBS4; AFP and UBE2T; HMMR and NXPH4; HMMR and PITX1; HMMR and THBS4; HMMR and UBE2T; NXPH4 and PITX1; NXPH4 and THBS4; NXPH4 and UBE2T; PITX1 and THBS4; PITX1 and UBE2T; THBS4 and UBE2T; AFP, HMMR and NXPH4; AFP, HMMR and PITX1; AFP, HMMR and THBS4; AFP, HMMR and UBE2T; AFP, NXPH4 and PITX1; AFP, NXPH4 and THBS4; AFP, NXPH4 and UBE2T; AFP, PITX1 and

THBS4; AFP, PITX1 and UBE2T; AFP, THBS4 and UBE2T; HMMR, NXPH4 and PITX1; HMMR, NXPH4 and ; HMMR, NXPH4 and THBS4; HMMR, NXPH4 and UBE2T; HMMR, PITX1 and THBS4; HMMR, PITX1 and UBE2T; HMMR, THBS4 and UBE2T; NXPH4, PITX1 and THBS4; NXPH4, PITX1 and UBE2T; and NXPH4, THBS4 and UBE2T.

In one aspect, the present disclosure relates to a method for screening an anticancer candidate substance, the method including steps of (a) measuring an expression level of AFP, HMMR, NXPH4, PITX1, THBS4, or UBE2T gene in liver cancer cells; (b) administering an anticancer candidate substance to the liver cancer cells and measuring an expression level of AFP, HMMR, NXPH4, PITX1, THBS4 or UBE2T gene in the liver cancer cells; and (c) when the expression level of AFP, HMMR, NXPH4, PITX1, THBS4 or UBE2T gene in the step (b) is lower than the expression level of AFP, HMMR, NXPH4, PITX1, THBS4 or UBE2T gene in the step (a), determining the anticancer candidate substance as an effective anticancer substance.

In one aspect, the present disclosure relates to a method for providing information necessary for diagnosis of liver cancer, the method including steps of (a) measuring an expression level of one or more biomarker genes selected from a group consisting of AFP, HMMR, NXPH4, PITX1, THBS4 and UBE2T in a biological sample isolated from a test subject; (b) measuring an expression level of the one or more biomarker genes in a normal control group sample; and (c) when the expression level of the biomarker gene in the step (a) is higher than the expression level of the biomarker gene in the step (b), determining that the test subject has liver cancer.

In one embodiment, the method may further include a step of distinguishing early liver cancer and advanced liver cancer from each other based on expression level change of the biomarker gene.

In one embodiment, a specific method of measuring the expression level of the mRNA of the biomarker gene or the protein thereof may detect the expression of the gene at the mRNA level or protein level. Separation of mRNA or protein from the biological sample may be performed using a known process. The gene expression level may be identified via a reverse transcriptase-polymerase chain reaction or a real time-polymerase chain reaction.

In one embodiment, the biological sample may include a sample, etc. such as tissue, cells, whole blood, serum, plasma, saliva, sputum, cerebrospinal fluid or urine, and the like. More preferably, the sample is whole blood, serum or plasma.

The present disclosure will be described in more detail based on following examples. However, the following examples are only for specifying the content of the present disclosure, and the present disclosure is not limited thereto.

### Example 1. Blood Marker Screening Using Database

### 1-1. HCC-Specific Marker Screening

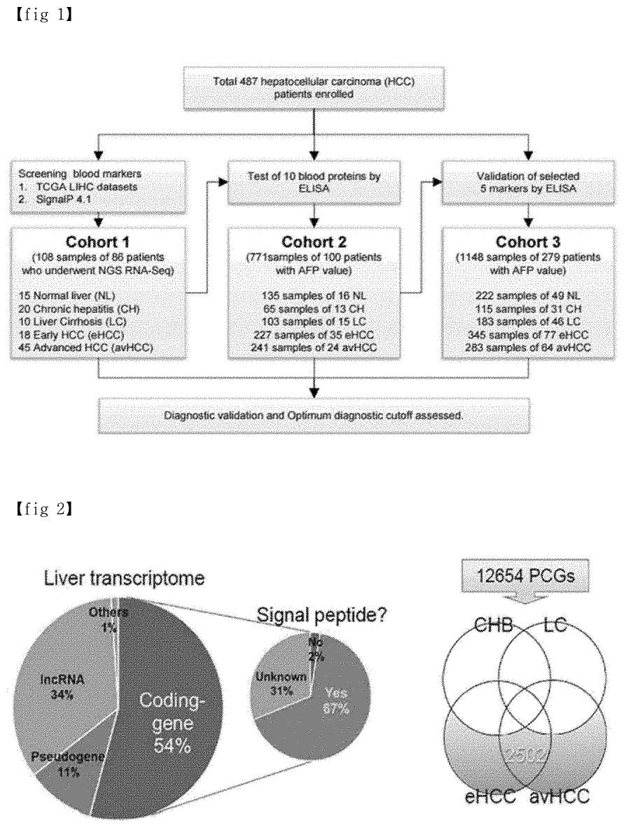

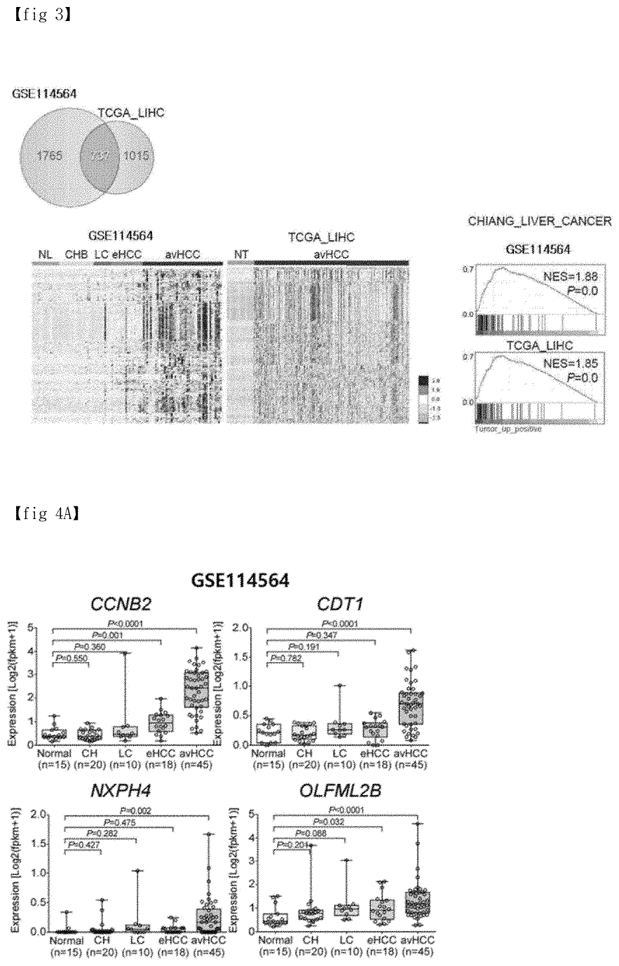

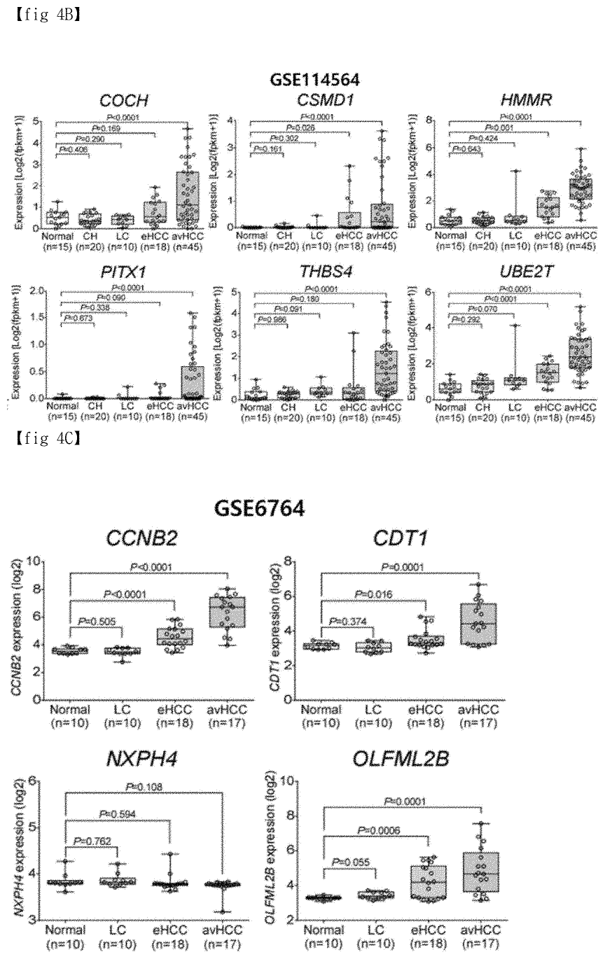

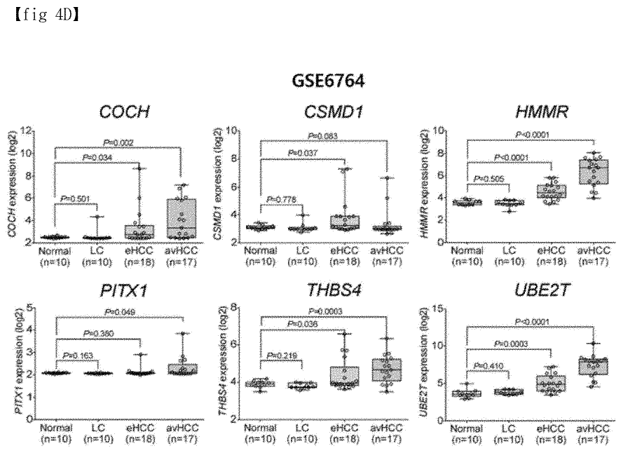

We selected 3 independent sets from blood samples (15 patient samples from normal liver (Normal liver, NL); 20 patient samples from chronic hepatitis (Chronic hepatitis, CH); 10 patient samples from cirrhosis (Liver Cirrhosis, LC); 18 patient samples from early hepatocellular carcinoma (Early HCC, eHCC); and 45 patient samples from advanced hepatocellular carcinoma (Advanced HCC, avHCC)) from independent liver disease patient cohort (108 samples from 86 patients), and then extracted total RNA therefrom using TRIzol reagent. A sequencing library was prepared using RNA Library Prep Kit for Illumina (Cat #E7420L), and sequencing was performed with Illumina HiSeq 2000 according to the standard method of Illumina. We mapped the entire transcriptome of the analyzed liver using STAR and Gencode v.25. Then, the expression profile was replaced with the FPKM value, and a gene type thereof was classified with Gencode v.25. 12,654 signal peptides were derived with SignalP 4.1. All data produced was registered in GEO as an open Omix database. Then, we derived 2,502 gene-elements having transmembrane domain that were overexpressed, and that are secretory peptide of protein in hepatocellular carcinoma (FIG. 2) and analyzed the 2,502 gene elements using Cancer Genome Atlas hepatocellular carcinoma (TCGA_LIHC) data and Gene Expression Omnibus (GEO) database. Then, 737 genes overexpressed in both databases were subjected to hierarchical clustering analysis. As a result, the GSE114564 database exhibited a system diagram divided into five distinct subclusters: normal liver, chronic hepatitis (CHB), cirrhosis, early hepatocellular carcinoma and advanced hepatocellular carcinoma. The TCGA_LIHC database exhibited a system diagram divided into two distinct subclusters of normal liver and advanced hepatocellular carcinoma (FIG. 3). When comparing the expression patterns of the calculated 737 genes in the two data sets, a distinct difference in expression change in advanced liver cancer was identified compared to the normal liver tissue. Further, for the analysis of gene data having two classes, gene set enrichment analysis (GSEA) to extract significant gene-sets exhibiting statistically significant differences in expression values of two classes among various gene-sets composed based on biological characteristics was performed. Thus, it could be identified that there is a very close correlation with the CHANG_LIVER_CANCER data set as one of the existing well-known liver cancer cohort gene sets. (Mean aggregation index NES=1.88, NES=1.85). Then, we identified 10 candidate marker genes that appear to be gradually overexpressed in the development of multistage liver cancer in both the GSE114564 and GSE6764 data cohort (FIGS. 4A-4D)

### 1-2. Individual Validation of Normal Liver and Advanced Hepatocellular Carcinoma Using TCGA LIHC and ICGC LIRI

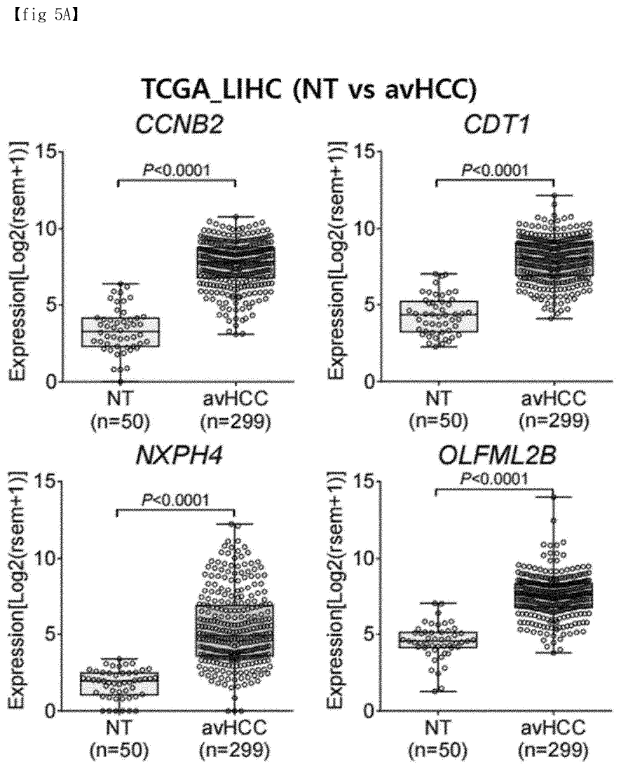

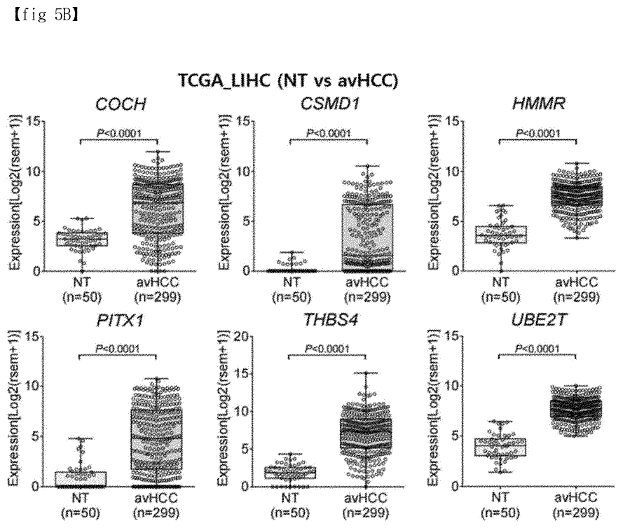

In order to implement a biomarker whose expression increases significantly in the patient's serum, the expression increase in advanced liver cancer must be statistically significantly higher than that in normal liver. Thus, we analyzed each of differential gene expressions of candidates 10 marker genes in HCC patients with corresponding non-cancer derived from sequencing-based data set allowing accurate expression measurement, and TCGA_LIHC data set and ICGC_LIRI data set as large-scale cohorts among public data. Thus, it was identified that both cohorts had marked difference in expression (FIGS. 5A-5D).

### 1-3. HCC Patient's 50-Matched Pair Analysis

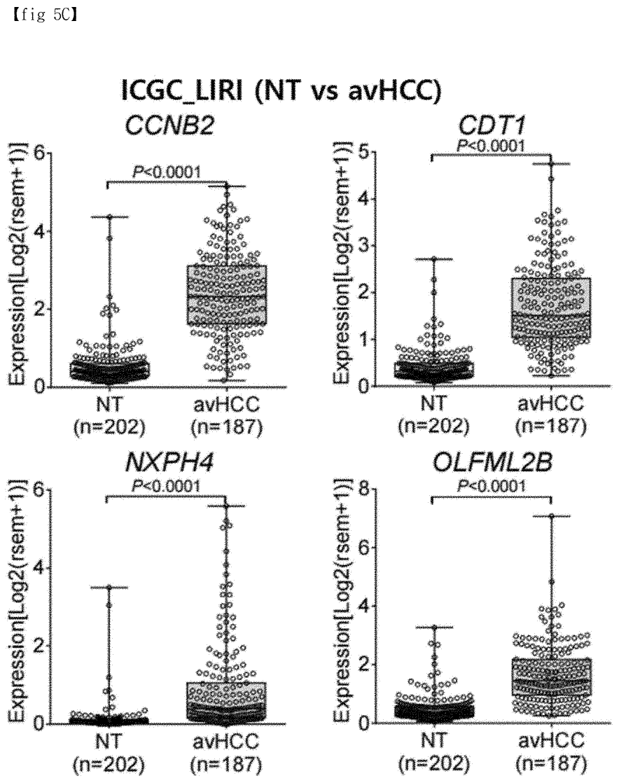

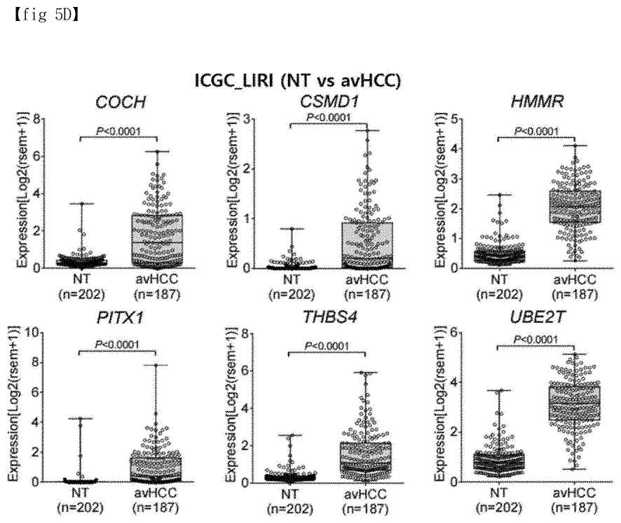

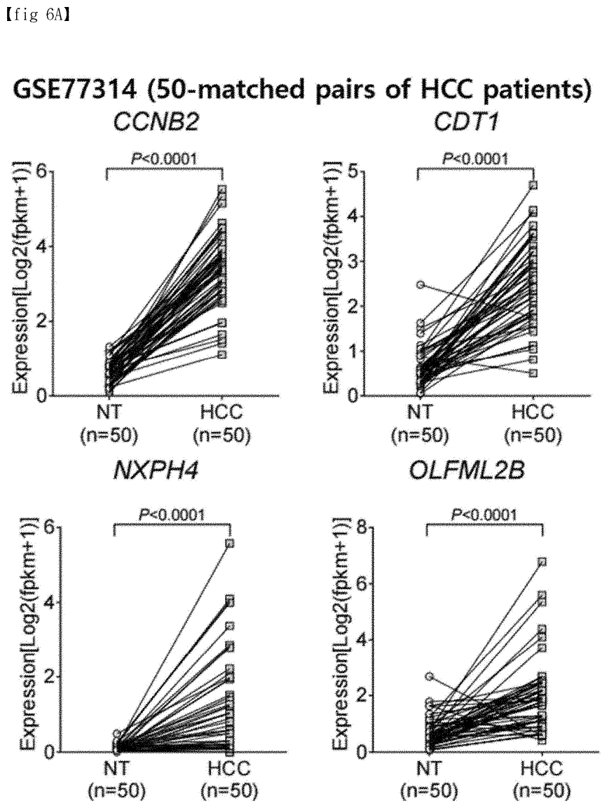

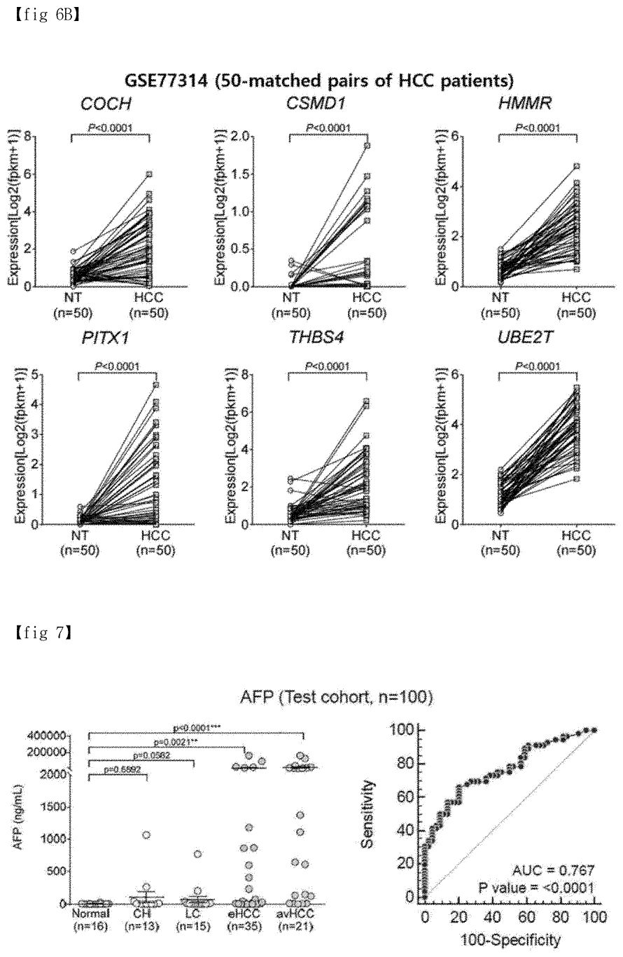

Based on a result of comparing and analyzing the expression levels of the 10 marker genes using the GSE77314 data set whose gene expression values were obtained via sequencing in the surrounding normal tissues and liver cancer tissues of a total of 50 liver cancer patients as a cohort of Chinese liver cancer patients, it was identified that in most of the patients, the expression was significantly increased in liver cancer tissues compared to normal liver tissues (FIGS. 6A-6B).

### Example 2. ELISA Analysis of First Selected Markers

### 2-1. Marker Expression Profile Identification

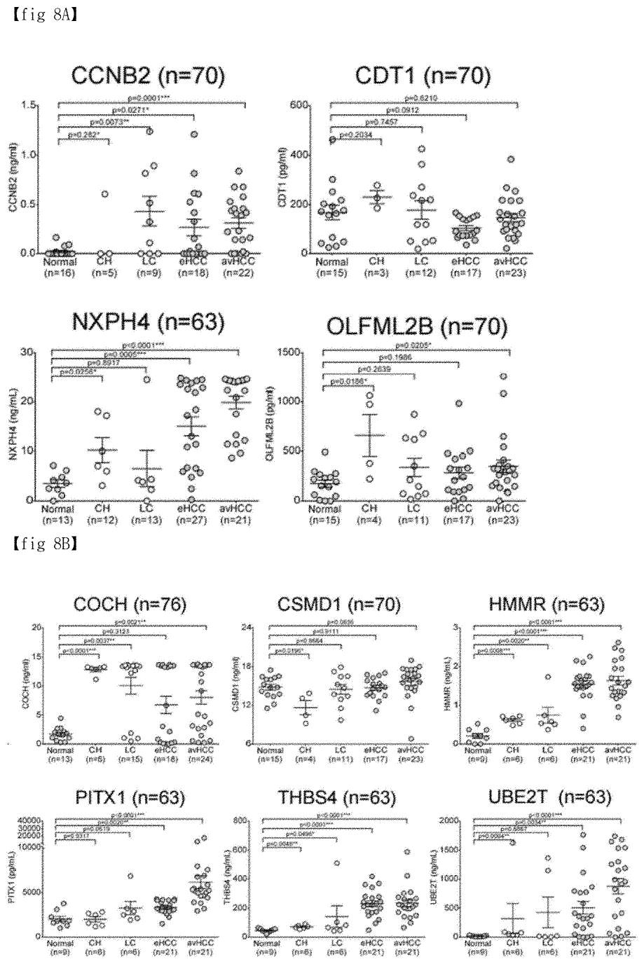

The expression levels of the 10 marker genes selected in Example 1 were identified via ELISA analysis in blood samples (135 samples of 16 patients with normal liver (Normal liver, NL); 65 samples of 13 patients with chronic hepatitis (Chronic hepatitis, CH); 103 samples of 15 patients with cirrhosis (Liver Cirrhosis, LC); 227 samples of 35 patients with early hepatocellular carcinoma (Early HCC, eHCC); and 241 samples of 24 patients with advanced hepatocellular carcinoma (Advanced HCC, avHCC) from independent liver disease patient cohort (771 samples from 100 patients) whose AFP values as HCC cancer markers were identified) (FIG. 7) (FIGS. 8A-8B).

As a result, normal liver patient (NL) had an average of 0.02 ng/ml of CCNB2. Chronic hepatitis patient (CH) had 0.2029 ng/ml thereof, cirrhosis patient (LC) had 0.43 ng/ml, early hepatocellular carcinoma patient (eHCC) had 0.27 ng/ml thereof and advanced hepatocellular carcinoma patient (avHCC) had 0.31 ng/ml thereof Thus, LC has the highest value of CCNB2. The patient (NL) of normal liver had an average of 167.7 pg/ml of CDT1, CH had 230.8 pg/ml thereof, LC had 178.2 pg/ml thereof, eHCC had 103.5 pg/ml thereof and avHCC had 146.8 pg/ml thereof. Thus, avHCC had the highest value of CDT1. Overall, there was no significant difference between diseases. The patient (NL) of normal liver had an average of 1.724 ng/ml of COCH, CH had 12.78 ng/ml thereof, LC had 10.03 ng/ml thereof, eHCC had 6.74 ng/ml thereof and avHCC had 8.025 ng/ml thereof. Thus, all stages of liver disease and liver cancer except normal liver had the higher level of COCH. The patients in normal liver (NL) had an average of 14.8 ng/ml of CSMD1, CH had 11.65 ng/ml thereof, LC had 14.48 ng/ml thereof, eHCC had 14.72 ng/ml thereof and avHCC had 15.66 ng/ml thereof. Thus, the levels were generally similar for CSMD1. The NL had an average of 175.2 pg/ml of OLFML2B, CH had 658.8 pg/ml thereof, LC had 338.4 pg/ml thereof, eHCC had 284.1 pg/ml thereof and avHCC had 349.6 pg/ml thereof. Thus, all liver diseases and liver cancer stages other than normal liver had high levels of OLFML2B. Especially, CH exhibited the high level of OLFML2B. NL had an average of 0.21 ng/ml of HMMR, CH had 0.62 ng/ml thereof, LC had 0.74 ng/ml thereof, eHCC had 1.54 ng/ml thereof and avHCC had 1.64 ng/ml thereof. In a similar manner to the sequencing results, the level of HMMR increased as the liver disease stage progressed. NL had an average of 3.54 ng/ml of NXPH4, CH had 10.23 ng/ml thereof, LC had 6.52 ng/ml thereof, eHCC had 15.02 ng/ml and avHCC had 19.83 ng/ml thereof. Thus, NXPH4 level was somewhat higher in CH. However, in the similar manner to the sequencing results, the level of NXPH4 increased as the liver disease stage progressed. PITX1 level was an average of 2,042 pg/ml in NL, 1,994 pg/ml in CH, 3,238 pg/ml in LC, 3,314 pg/ml in eHCC and 6,135 pg/ml in avHCC. PITX1 level was lower in CH than that in normal liver. However, in the similar manner to the sequencing results, the level of PITX1 increased as the liver disease stage progressed. NL had 45.36 ng/ml of THBS4, CH had 70.96 ng/ml thereof, LC had 141.8 ng/ml, eHCC had 229.4 ng/ml and avHCC had 233.6 ng/ml thereof In the similar manner to the sequencing results, the level of THBS4 increased as the liver disease stage progressed. The NL has average 16.14 ng/ml of UBE2T, CH had 319.9 ng/ml thereof, LC had 426.1 ng/ml thereof, eHCC had 505.5 ng/ml thereof and avHCC had 877.2 ng/ml thereof The UBE2T level was about 20 times higher in liver disease than the normal liver. The level of UBE2T increased as the liver disease stage progressed.

### 2-2. ROC (Receiver Operating Characteristic) Curve Analysis

ROC curve analysis was performed with ELISA values for 10 marker genes in the cohort.

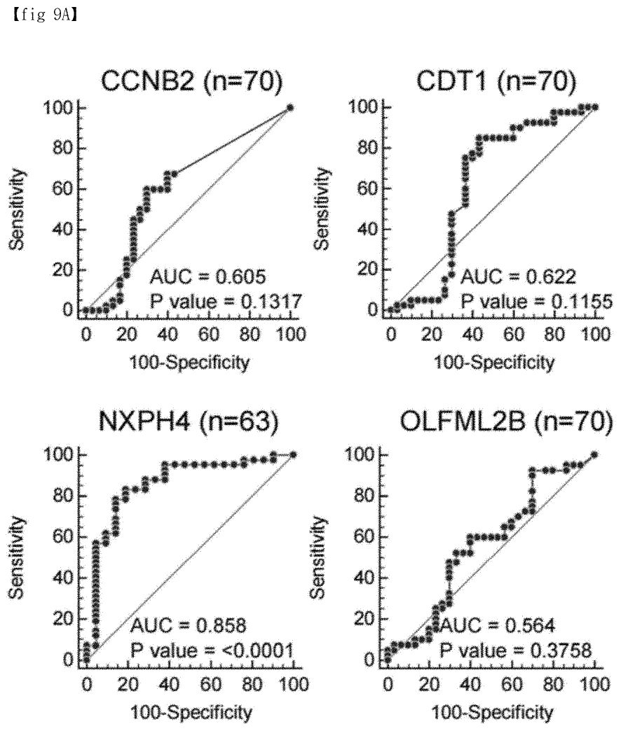

As a result, it was found that CSMD1, HMMR, NXPH4, OPITX1, THBS4 and UBE2T had statistically significant values compared to the reference line. ROC curve analysis exhibited that HMMR, NXPH4, PITX1, THBS4 and UBE2T had AUC (area under the curve) values similar to or higher than that of AFP, thus indicating that the markers HMMR, NXPH4, PITX1, THBS4 and UBE2T had specificity and sensitivity (FIGS. 9A-9B).

### Example 3. Validation of Early Cancer Diagnostic Markers HMMR, NXPH4, PITX1, THBS4 and UBE2T

### 3-1. Marker Expression Profile Identification

The expression levels of 10 marker genes selected in Example 1 were identified via ELISA analysis in blood samples (222 samples from 49 patients with normal liver (Normal liver, NL); 115 samples of 31 patients with chronic hepatitis (Chronic hepatitis, CH); 183 samples of 46 patients with cirrhosis (Liver Cirrhosis, LC); 345 samples of 77 patients with early hepatocellular carcinoma (Early HCC, eHCC); and 283 samples of 64 patients with advanced hepatocellular carcinoma (Advanced HCC, avHCC) from independent liver disease patient cohort (1,148 samples from 279 patients) whose AFP values as HCC cancer markers were identified) (FIG. 10) (FIGS. 11A-11B).

We measured the protein expression level of each of the 5 markers in the validation cohort. Based on a result of comparing the normal group with each liver disease group in a total of 230 samples, it was found that there was a very significant difference in the HMMR level except for the cirrhosis group. In particular, it was identified that HMMR was specifically highly expressed in early liver cancer. The expression level change of NXPH4 was higher in all liver disease stage groups than in the normal group. PITX1 also had similar results. As in HMMR, THBS4 level significantly increased except for the cirrhosis group, and was high in the early liver cancer group. As in the test cohort, the expression of UBE2T did not occur at all in the normal group, and the expression of UBE2T was increased in the liver disease stage group.

### 3-2. ROC Curve Analysis

ROC curve analysis was performed for marker genes HMMR, NXPH4, PITX1, THBS4 and UBE2T in the cohort.

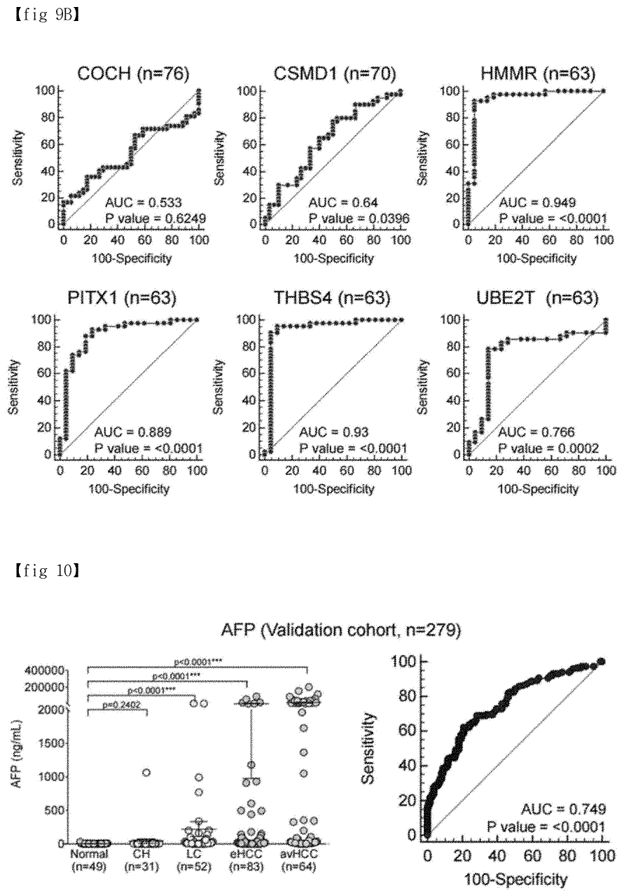

As a result, HMMR and THBS4 had values of AUC=0.856 and AUC=0.772, respectively. Thus, it was identified that the levels thereof were higher than AUC=0.749 of the existing marker AFP (FIGS. 12A-12B).

### 3-3. Identification of Expression Patterns in Stages of Development of Hepatocellular Carcinoma

In order to identify the sensitivity, specificity and accuracy of HMMR, NXPH4, PITX1, THBS4 and UBE2T in the cohort validated by AFP, ELISA analysis of the cohort sample was performed.

TABLE 1

| | | | | | | |
|---|---|---|---|---|---|---|
| AUC | Sensi- | Speci- | | | | |
| (95% | tivity | ficity | Accuracy | PPV | NPV | Odds |
| CI) | (%) | (%) | (%) | (%) | (%) | ratio |

HCC vs CHB, LC and HC

| | | | | | | | |
|---|---|---|---|---|---|---|---|
| AFP | 0.793 | 51.13 | 86.05 | 64.84 | 85 | 53.24 | 6.45 |
| HMMR | 0.914 | 79.7 | 91.86 | 84.47 | 93.81 | 74.53 | 44.31 |
| NXPH4 | 0.789 | 74.44 | 74.42 | 74.43 | 81.82 | 65.31 | 8.47 |
| PITX1 | 0.777 | 79.7 | 63.95 | 73.52 | 77.37 | 67.07 | 6.97 |
| THBS4 | 0.791 | 57.14 | 88.37 | 69.41 | 88.37 | 57.14 | 10.13 |
| UBE2T | 0.624 | 59.4 | 69.77 | 63.47 | 75.24 | 52.63 | 3.38 |

HCC vs CHB, LC

| | | | | | | | |
|---|---|---|---|---|---|---|---|
| AFP | 0.717 | 51.13 | 71.79 | 55.81 | 86.08 | 30.11 | 2.66 |
| HMMR | 0.832 | 79.7 | 82.05 | 80.23 | 93.81 | 54.24 | 17.95 |
| NXPH4 | 0.694 | 74.44 | 51.28 | 69.19 | 83.9 | 37.04 | 3.07 |
| PITX1 | 0.718 | 79.7 | 48.72 | 72.67 | 84.13 | 41.3 | 3.73 |
| THBS4 | 0.735 | 57.14 | 79.49 | 62.21 | 90.48 | 35.23 | 5.17 |
| UBE2T | 0.575 | 59.4 | 33.33 | 53.49 | 75.24 | 19.4 | 0.73 |

eHCC vs CHB, LC and HC

| | | | | | | | |
|---|---|---|---|---|---|---|---|
| AFP | 0.71 | 31.43 | 86.05 | 61.54 | 64.71 | 60.66 | 2.83 |
| HMMR | 0.915 | 81.43 | 91.86 | 87.18 | 89.06 | 85.87 | 49.48 |
| NXPH4 | 0.742 | 62.86 | 74.42 | 69.23 | 66.67 | 71.11 | 4.92 |
| PITX1 | 0.681 | 71.43 | 63.95 | 67.31 | 61.73 | 73.33 | 4.44 |
| THBS4 | 0.748 | 52.86 | 88.37 | 72.44 | 78.72 | 69.72 | 8.52 |
| UBE2T | 0.591 | 52.86 | 69.77 | 62.18 | 58.73 | 64.52 | 2.59 |

eHCC vs CHB, LC

| | | | | | | | |
|---|---|---|---|---|---|---|---|
| AFP | 0.613 | 31.43 | 71.79 | 45.87 | 66.67 | 36.84 | 1.17 |
| HMMR | 0.835 | 81.43 | 82.05 | 81.65 | 89.06 | 71.11 | 20.04 |
| NXPH4 | 0.648 | 62.88 | 51.28 | 58.72 | 69.84 | 43.48 | 1.78 |
| PITX1 | 0.606 | 71.43 | 48.72 | 63.3 | 71.43 | 48.72 | 2.38 |
| THBS4 | 0.693 | 52.86 | 79.49 | 62.39 | 82.22 | 48.44 | 4.34 |
| UBE2T | 0.622 | 52.86 | 33.33 | 45.87 | 58.73 | 28.26 | 0.56 |

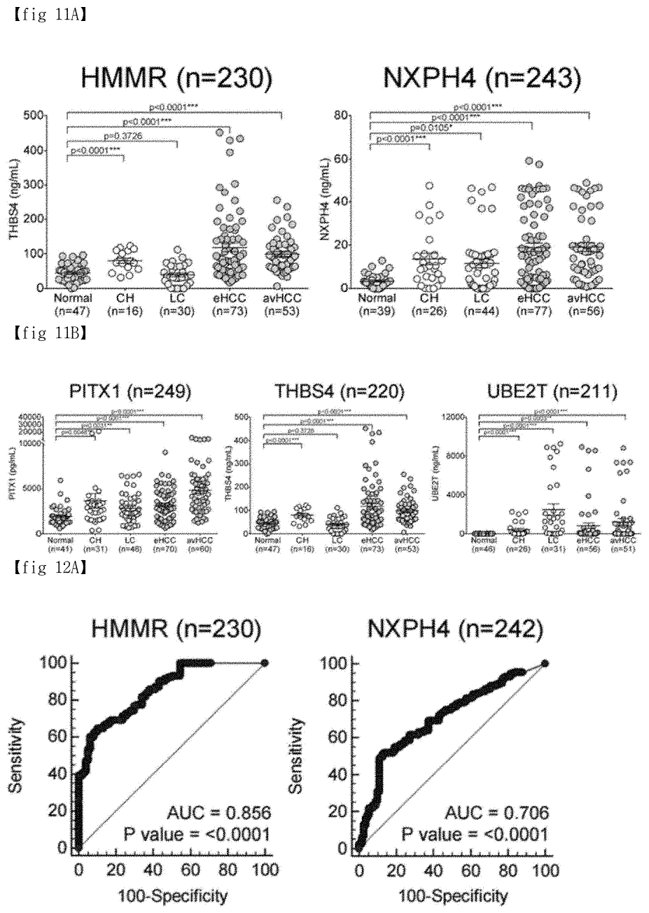

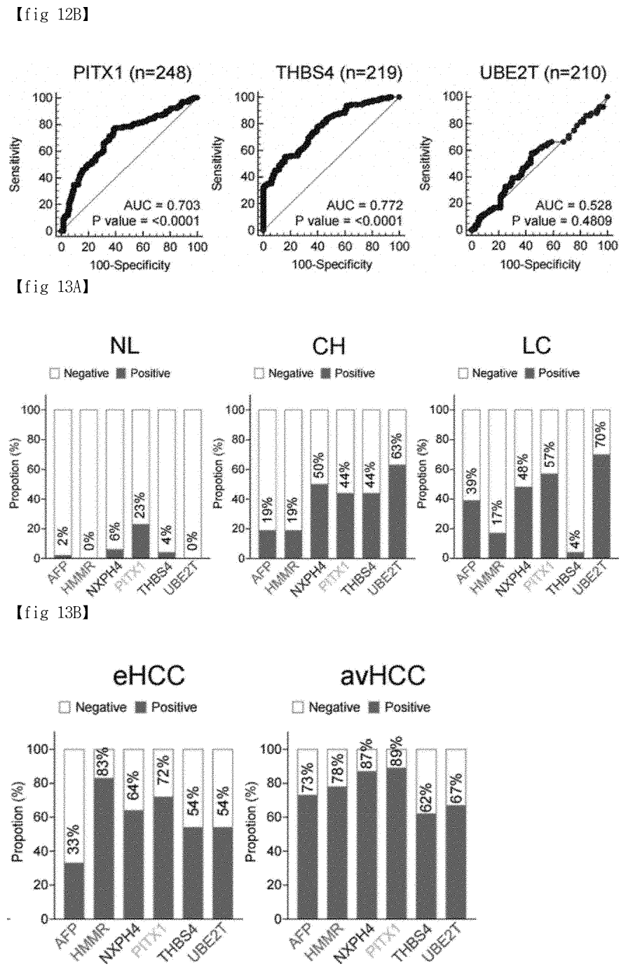

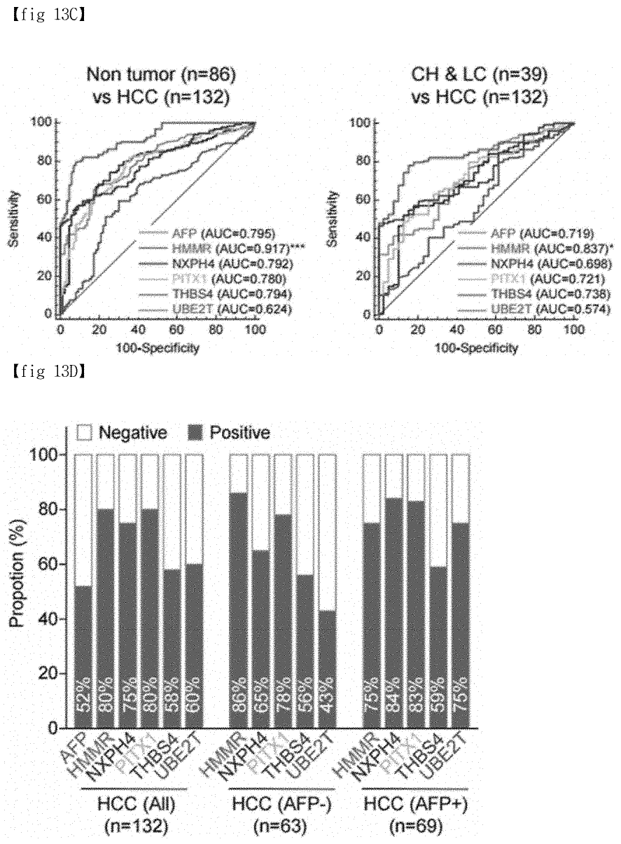

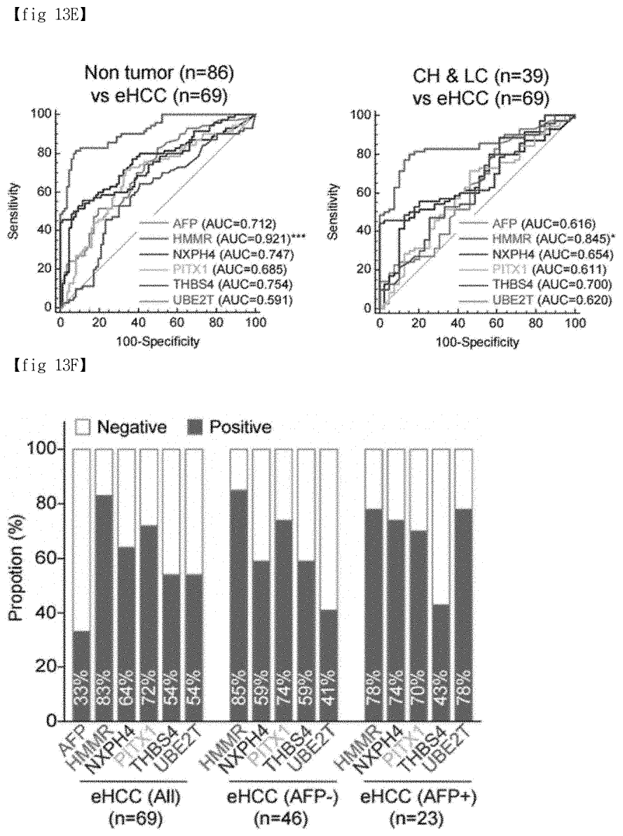

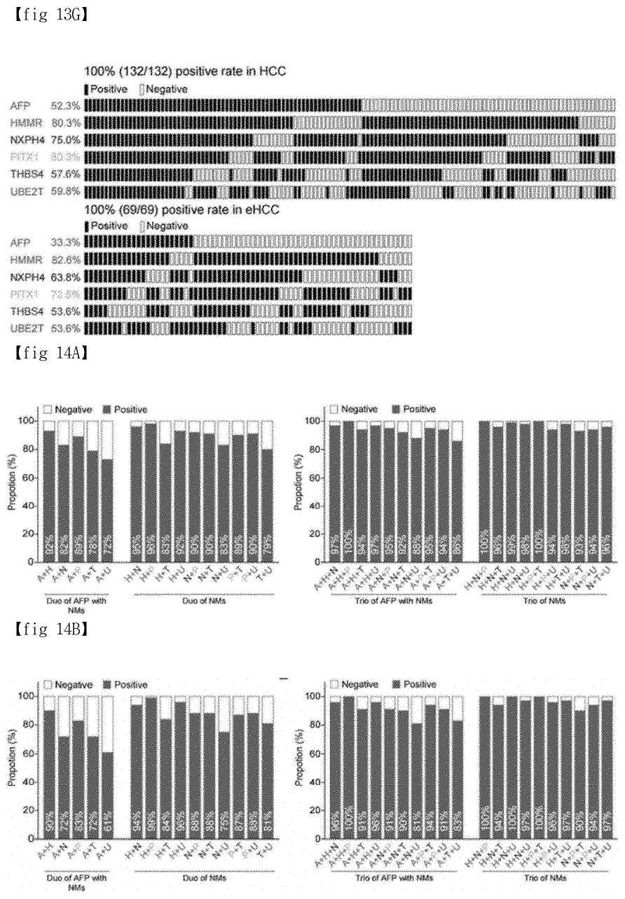

The result is shown in Table 1 and FIGS. 13A-13G. Specifically, for 5 markers along with AFP, 1) non-liver cancer samples (normal liver, hepatitis, cirrhosis samples) and liver cancer samples were compared with each other, 2) liver disease sample (hepatitis, cirrhosis sample) and liver cancer sample were compared with each other, and then 5 markers along with AFP were analyzed specifically to early liver cancer, and in 3) non-liver cancer samples and 4) liver disease samples, respectively. In all four cases, HMMR exhibited the highest sensitivity, specificity and accuracy. When calculating the cut-off values of each of these markers using the MedCal program, HMMR had 0.8 ng/μl of the cut-off value, NXPH4 had 7.5 ng/μl thereof, PITX1 had 2,475 pg/μl thereof, THBS4 had 90 ng/μl thereof and UBE2T had 40 ng/μl thereof. When the values of the samples increased above the cut-off value, the samples were analyzed as positive. When the values of samples decreased below the cut-off value, the samples were analyzed as negative. In the normal liver, a positive percentage for AFP was 2%, a positive percentage for HMMR was 0%, a positive percentage for NXPH4 was 6%, a positive percentage for PITX1 was 23%, a positive percentage for THBS4 was 4% and a positive percentage for UBE2T was 0%. In the hepatitis group, a positive percentage for AFP was 19% and a positive percentage for HMMR was 19%. A positive percentage for NXPH4 was 50%, a positive percentage for PITX1 was 44%, a positive percentage for THBS4 was 44% and a positive percentage for UBE2T was 63%. In the cirrhosis group, a positive percentage for AFP was 39%, a positive percentage for HMMR was 17%, a positive percentage for NXPH4 was 48%, a positive percentage for PITX1 was 57%, a positive percentage for THBS4 was 4% and a positive percentage for UBE2T was 70%. In the early liver cancer group, a positive percentage for AFP was 33%, a positive percentage for HMMR was 83%, NXPH4 was 64%, a positive percentage for PITX1 was 72%, a positive percentage for THBS4 was 54%, and a positive percentage for UBE2T was 54%, and thus, the 5 markers were measured at significantly higher positive percentages than AFP, a marker for measuring liver cancer. In the advanced liver cancer group, a positive percentage for AFP was 73%, a positive percentage for HMMR was 78%, a positive percentage for NXPH4 was 87%, a positive percentage for PITX1 was 89%, a positive percentage for THBS4 was 62% and a positive percentage for UBE2T was 67%. Next, when comparing positive percentages for AFP and the 5 markers in liver cancer patient, a positive percentage for AFP was 52%, while positive percentages for the other markers were high. In particular, when comparing positive percentages for the five markers in liver cancer patients with a negative percentage for AFP, a positive percentage for HMMR was 86%. Thus, HMMR is expected to complement a test result for liver cancer patients whose AFP is not measured as positive. In the case of early liver cancer group, a positive percentage for AFP was 33%, while a positive percentage for HMMR was 83%. In addition, a positive percentage for HMMR was 85% in liver cancer patients whose AFP is measured as negative.

### Example 4. Identification of Effect of Combinations of Early Cancer Diagnostic Markers

For the cohort of Example 3-1, the diagnostic effect of hepatocellular carcinoma was analyzed based on a combination of two markers among AFP, HMMR, NXPH4, PITX1, THBS4 and UBE2T (combination of AFP and HMMR, NXPH4, PITX1, THBS4 or UBE2T; combinations of two of HMMR, NXPH4, PITX1, THBS4 and UBE2T) or a combination of three markers thereof (combinations of AFP and two markers among HMMR, NXPH4, PITX1, THBS4 and UBE2T; or combinations of three markers of HMMR, NXPH4, PITX1, THBS4 and UBE2T).

TABLE 2
HCC vs Non tumor (Normal, CHS and LC)

| | | | Sensitivity | Specificity | | | Accuracy | PPV | NPV | Odds | Relative |
|---|---|---|---|---|---|---|---|---|---|---|---|
| | AUC | 95% Cl | (%) | (%) | +LR | −LR | (%) | (%) | (%) | ratio | risk |
| AFP | 0.795 | 0.735-0.846 | 52.27 | 84.08 | 8.48 | 0.58 | 65.14 | 53.37 | 84.86 | 6.55 | 0.71 |
| AFP-HMMR | 0.945 | 0.907-0.972 | 80.18 | 80.37 | 7.75 | 0.11 | 89.45 | 62.28 | 88.39 | 69.59 | 3.33 |
| HMMR-NXPH4 | 0.938 | 0.897-0.968 | 78.78 | 93.38 | 8.88 | 0.23 | 88.94 | 83.84 | 22.63 | 49.92 | 8.52 |
| HMMR-PITX1 | 0.045 | 0.908-0.971 | 92.42 | 98.08 | 8.02 | 0.00 | 88.31 | 91.42 | 36.20 | 95.28 | 7.05 |
| HMMR-UBE2T | 0.025 | 0.882-0.987 | 87.98 | 83.72 | 6.42 | 0.34 | 86.24 | 87.88 | 83.92 | 97.25 | 4.92 |
| AFP-HMMR-PITX1 | 0.088 | 0.024-0.079 | 83.04 | 84.38 | 6.21 | 0.07 | 90.37 | 93.53 | 54.81 | 87.54 | 10.90 |
| HMMR-NXPH4-PITX1 | 0.950 | 0.012-0.918 | 92.42 | 88.95 | 8.82 | 0.08 | 33.31 | 92.42 | 86.03 | 75.21 | 7.95 |
| HMMR-NXPH4-UBE2T | 0.943 | 0.825-0.968 | 81.87 | 21.46 | 4.33 | 0.10 | 87.81 | 87.81 | 84.43 | 48.33 | 2.57 |
| HMMR-PITX1-THBS4 | 0.948 | 0.907-0.092 | 90.83 | 83.72 | 6.80 | 0.31 | 88.07 | 90.85 | 83.72 | 34.43 | 6.52 |

eHCC vs Non tumor (Normal, CHS and LC)

| | | | Sensitivity | Specificity | | | Accuracy | PPV | NPV | Odds | Relative |
|---|---|---|---|---|---|---|---|---|---|---|---|
| | AUC | 95% Cl | (%) | (%) | +LR | −LR | (%) | (%) | (%) | ratio | risk |
| AFP | 0.712 | 0.834-0.982 | 23.83 | 84.88 | 2.21 | 0.79 | 61.94 | 85.53 | 54.85 | 2.81 | 8.62 |
| AFP-HMMR | 0.231 | 0.574-0.865 | 80.80 | 85.37 | 7.45 | 0.55 | 87.78 | 86.98 | 58.37 | 50.07 | 8.23 |
| HMMR-NXPH4 | 0.934 | 0.852-0.967 | 81.98 | 93.06 | 9.37 | 0.23 | 87.36 | 85.36 | 91.88 | 48.82 | 8.35 |
| HMMR-PITX1 | 0.838 | 0.898-0.960 | 91.86 | 88.05 | 8.56 | 0.40 | 88.88 | 81.30 | 86.05 | 84.75 | 13.88 |
| HMMR-UBE2T | 0.073 | 0.854-0.988 | 82.81 | 88.52 | 7.89 | 0.36 | 86.43 | 82.61 | 88.53 | 40.64 | 5.92 |
| AFP-HMMR-PITX1 | 0.841 | 0.892-0.973 | 92.75 | 83.72 | 5.75 | 0.00 | 87.74 | 92.75 | 53.72 | 85.03 | 15.95 |
| HMMR-NXPH4-PITX1 | 0.838 | 0.888-0.971 | 91.20 | 80.08 | 6.54 | 0.10 | 88.30 | 91.30 | 86.05 | 53.75 | 15.03 |
| HMMR-NXPH4-UBE2T | 0.838 | 0.888-0.969 | 84.05 | 58.37 | 7.23 | 0.18 | 85.45 | 84.66 | 88.37 | 42.02 | 8.37 |
| HMMR-PITX1-THBS4 | 0.836 | 0.885-0.969 | 88.88 | 83.72 | 5.52 | 0.42 | 85.45 | 88.56 | 43.72 | 45.65 | 11.04 |

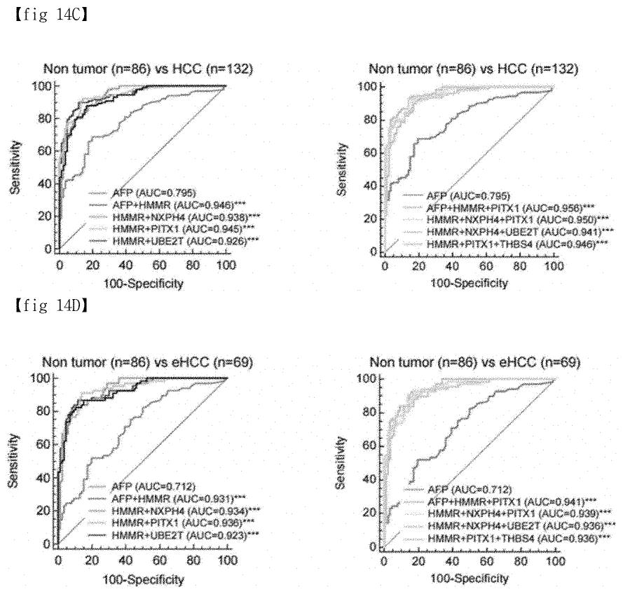

As a result, the results were shown in Table 2 and FIGS. 14A-14D. Specifically, when the two markers were combined with each other, when targeting all liver cancer patients, the combination of AFP and HMMR exhibited a positive percentage of 92%. The combination of HMMR and PITX1 had the highest positive percentage of 96%. When targeting patients with early liver cancer, the combination of AFP and HMMR exhibited a positive percentage of 90%. The combination of HMMR and PITX1 had the highest positive percentage of 99%. Further, when the three markers were combined with each other, and when targeting all liver cancer patients, the combination of AFP, HMMR and PITX1, the combination of HMMR, NXPH4 and PITX1, and the combination of HMMR, PITX1 and UBE2T had the highest positive percentage of 100%. When targeting patients with early liver cancer, the combinations of AFP, HMMR and PITX1, HMMR, NXPH4 and PITX1, HMMR, NXPH4 and UBE2T, and HMMR, PITX1 and UBE2T had the highest positive percentage of 100%.

In addition, when ROC analysis was performed on combinations showing a positive percentage of 100% in 86 non-liver cancer samples and 132 liver cancer samples, it was found that all combinations had significantly increased AUC values statistically than conventional AFP had. Among the combinations of the two markers, the combination of AFP and HMMR was evaluated as the best. Among the combinations of the three markers, the combination of AFP, HMMR and PITX1 exhibited the highest value. Regarding a diagnostic analysis, among the combinations of the two markers, the combination of HMMR and PITX1 exhibited the highest in the accuracy. The odds ratio thereof was also the highest value of 75.23. Among the combinations of the three markers, the combination of AFP, HMMR and PITX1 exhibited the highest accuracy of 90.37%. The odds ratio thereof was also the highest value of 87.04. Further, when ROC analysis was performed on combinations showing a positive percentage of 100% in 86 non-liver cancer samples and 69 early liver cancer samples, it was identified that the AUC value was significantly increased in all combinations statistically than that of the existing AFP. Among the combinations of the two markers, the combination of HMMR and PITX1 was evaluated as the best. Among the combinations of the three markers, the combination of AFP, HMMR and PITX1 exhibited the highest value. Regarding a diagnostic analysis, among the combinations of the two markers, the combination of HMMR and PITX1 exhibited the highest accuracy of 88.39%. The odds ratio thereof was also the highest value of 64.75. Among the combinations of the three markers, the combinations of AFP, HMMR and PITX1 exhibited the highest accuracy of 92.75%. The odds ratio thereof was also the highest value of 65.83.

1. A biomarker for diagnosis of liver cancer, the biomarker comprising at least one gene selected from a group consisting of AFP (α-fetoprotein), HMMR (hyaluronan-mediated motility receptor), NXPH4 (neurexophilin 4), PITX1 (paired-like homeodomain 1), THBS4 (thrombospondin 4) and UBE2T (ubiquitin-conjugating enzyme E2T) or a protein expressed from the at least one gene.

2. The biomarker of claim 1, wherein the liver cancer is hepatocellular carcinoma (HCC).

3. The biomarker of claim 2, wherein the hepatocellular carcinoma includes early hepatocellular carcinoma or advanced hepatocellular carcinoma.

4. A composition for diagnosis of liver cancer, the composition comprising an agent for measuring an expression level of one or more biomarker genes selected from a group consisting of AFP, HMMR, NXPH4, PITX1, THBS4 and UBE2T at an mRNA or protein level.

5. The composition of claim 4, wherein the composition includes an agent for measuring an expression level of one or more biomarker gene sets selected from a group consisting of AFP and HMMR, AFP and NXPH4, AFP and PITX1, AFP and THBS4, AFP and UBE2T, HMMR and NXPH4, HMMR and PITX1, HMMR and THBS4, HMMR and UBE2T, NXPH4 and PITX1, NXPH4 and THBS4, NXPH4 and UBE2T, PITX1 and THBS4, PITX1 and UBE2T, and THBS4 and UBE2T at the mRNA or protein level.

6. The composition of claim 4, wherein the composition includes an agent for measuring an expression level of one or more biomarker gene sets selected from a group consisting of AFP, HMMR and NXPH4; AFP, HMMR and PITX1; AFP, HMMR and THBS4; AFP, HMMR and UBE2T; AFP, NXPH4 and PITX1; AFP, NXPH4 and THBS4; AFP, NXPH4 and UBE2T; AFP, PITX1 and THBS4; AFP, PITX1 and UBE2T; AFP, THBS4 and UBE2T; HMMR, NXPH4 and PITX1; HMMR, NXPH4 and; HMMR, NXPH4 and THBS4; HMMR, NXPH4 and UBE2T; HMMR, PITX1 and THBS4; HMMR, PITX1 and UBE2T; HMMR, THBS4 and UBE2T; NXPH4, PITX1 and THBS4; NXPH4, PITX1 and UBE2T; and NXPH4, THBS4 and UBE2T at the mRNA or protein level.

7. The composition of claim 4, wherein the liver cancer is hepatocellular carcinoma.

8. The composition of claim 7, wherein the hepatocellular carcinoma includes early hepatocellular carcinoma or advanced hepatocellular carcinoma.

9. The composition of claim 4, wherein the agent for measuring the expression level of the biomarker gene at the mRNA level includes a nucleic acid sequence of the marker, a nucleic acid sequence complementary to the nucleic acid sequence, and a primer pair and/or a probe that specifically recognizes a fragment of the nucleic acid sequence and the complementary nucleic acid sequence.

10. The composition of claim 9, wherein the measurement is performed using a scheme selected from a group consisting of polymerase chain reaction, real-time RT-PCR, reverse transcription polymerase chain reaction, competitive polymerase chain reaction (competitive RT-PCR), nuclease protection assay (RNase, S1 nuclease assay), in situ hybridization, nucleic acid microarray, Northern blots, or DNA chip.

11. The composition of claim 4, wherein the agent for measuring the expression level of the biomarker gene at the protein level includes an antibody, an antibody fragment, an aptamer, an avidity multimer or peptidomimetics that specifically recognizes a full length of a protein of the maker or a fragment thereof.

12. The composition of claim 11, wherein the measurement is performed using a scheme selected from a group consisting of Western blot, ELISA (enzyme linked immunosorbent assay), radioimmunoassay (RIA), radioimmunodiffusion, immunoelectrophoresis, tissue immunostaining, immunoprecipitation assay, complement fixation assay, FACS, mass spectrometry, or protein microarray.

13. A liver cancer diagnostic kit comprising the composition of claim 4.

14. A method for providing information necessary for diagnosis of liver cancer, the method comprising steps of:
(a) measuring an expression level of at least one biomarker gene selected from a group consisting of AFP, HMMR, NXPH4, PITX1, THBS4 and UBE2T in a biological sample isolated from a test subject;
(b) measuring an expression level of the at least one biomarker gene in a normal control group sample; and
(c) when the expression level of the biomarker gene in the step (a) is higher than the expression level of the biomarker gene in the step (b), determining that the test subject has liver cancer.

15. The method of claim 14, wherein the biological sample is blood or serum.

16. The method of claim 15, wherein the liver cancer includes early hepatocellular carcinoma or advanced hepatocellular carcinoma.
